(19) United States
(12) Patent Application Publication
(10) Pub. No.: US 2023/0312445 A1
(43) Pub. Date: Oct. 5, 2023

(54) DEVICE FOR DISTILLATION DECOLORIZATION AND PURIFICATION OF ALCOHOL AND METHOD FOR PURIFICATION IN MALTOL PRODUCTION

(71) Applicant: ANHUI JINHE INDUSTRIAL CO., LTD., Chuzhou City (CN)

(72) Inventors: Yuean CHEN, Chuzhou City (CN); Jian ZHAN, Chuzhou City (CN); Qijun ZHANG, Chuzhou City (CN)

(21) Appl. No.: 18/207,734

(22) Filed: Jun. 9, 2023

Related U.S. Application Data
(63) Continuation of application No. PCT/CN2021/137214, filed on Dec. 10, 2021.

(30) Foreign Application Priority Data
Dec. 11, 2020 (CN) 202011451866.6

(51) Int. Cl. C07C 29/84; B01J 19/24; B01J 4/00; B01D 3/40; B01D 3/14; B01D 3/00; B01D 5/00; B01D 3/32

(52) U.S. Cl. CPC C07C 29/84; B01J 19/24; B01J 4/001; B01D 3/40; B01D 3/143; B01D 3/009; B01D 5/0063; B01D 3/322; B01J 2204/005

(57) ABSTRACT

Disclosed is a device for distillation decolorization and purification of alcohol in maltol production. The device comprises a rectification column, an alkali hydrolysis kettle and an alkali hydrolysis rectification column. An upper outlet of the rectification column is connected to an inlet of the alkali hydrolysis kettle, an outlet of the alkali hydrolysis kettle is connected to an inlet of the alkali hydrolysis rectification column, and an upper outlet of the alkali hydrolysis rectification column is connected to an inlet of a finished product tank. Further disclosed is a method for distillation decolorization and purification of alcohol in maltol production. A product processed by the solution of the present application has a high purity.

This application claims priority to a Chinese patent application with an application date of Dec. 11, 2020 and application number of 202011451866.6, entitled as device for distillation decolorization and purification of alcohol and method for purification in maltol production.

### TECHNICAL FIELD

The present invention relates to a device for distillation decolorization and purification of alcohol and a method for purification in maltol production.

### BACKGROUND

The production process of maltol is divided into Grignard section, chlorination section, sublimation section, crystallization section, and drying section. In the crystallization section, alcohol is mainly used to recrystallize the material to remove impurities and odors in the material so as to improve product quality. After replacement, the alcohol is recovered by being distilled in a distillation kettle. But, the quality of the distilled alcohol is not good, which affects the quality of the product, and increase in replacement efforts will cause the cost to rise.

Chinese patent document CN103570497B discloses an inorganic membrane recovering alcohol treatment method, comprising the following steps: (a) by preliminary distilling, obtaining the acidic alcohol to be treated; (b) after pumping acidic alcohol into a second reactor, adding alkali to neutralize it until the pH value reaches 9 to obtain neutral alcohol; (c) pumping the neutral alcohol into an evaporator to extract neutral gaseous hydrous alcohol; (d) dehydrating the gaseous hydrous alcohol through an inorganic membrane dehydration device, extracting dehydrated ethanol with moisture content below 0.01% to an alcohol product receiving tank. Chinese patent document CN205182700U discloses an alcohol recovery and reuse system, wherein one-time recovery of ethanol solvent used in process of alkalization, acidification, refining purification and drying is carried out by rectification column equipment to recovery simultaneously ethanol in both gaseous and liquid forms. However, the above technology still has the problem of high cost during use.

### SUMMARY

The first technical problem to be solved by this invention is to provide a device for distillation decolorization and purification of alcohol in maltol production, which is simple in structure, easy to use and has low cost in use.

The second technical problem to be solved by this invention is to provide a purification method using above device for distillation decolorization and purification of alcohol in maltol production to obtain alcohol with high purity and less impurity.

For solving above-mentioned first technical problem, the invention provides a device for distillation decolorization and purification of alcohol in maltol production comprising a first feed pump, a rectification column, a first condenser, a reflux tank, a first reflux pump, a first reboiler, a first extraction pump, an alkali hydrolysis kettle, a second condenser, an alkali hydrolysis receiving tank, a second feed pump, an alkali hydrolysis rectification column, a third condenser, a finished product tank, a second reflux pump, a second reboiler, and a second extraction pump. The first feed pump is connected to an inlet of the rectification column, the first condenser is connected to an upper outlet of the rectification column, the other end of the first condenser is connected to an inlet of the reflux tank, an outlet of the reflux tank is connected to the rectification column through the first reflux pump, a lower part of the rectification column is connected to the first reboiler through a circulation pipeline, bottom of the rectification column is connected to the first production pump, an outlet of the reflux tank is connected to an inlet of the alkali hydrolysis kettle through the other pipeline, the alkali hydrolysis kettle is connected to the second condenser through a circulation pipeline, an outlet of the alkali hydrolysis kettle is connected to an inlet of the alkali hydrolysis receiving tank, an outlet of the alkali hydrolysis receiving tank is connected to an inlet of the alkali hydrolysis rectification column through the second feed pump, an upper outlet of the alkali hydrolysis rectification column is connected to the third condenser, the other end of the third condenser is connected to an inlet of the finished product tank, an outlet of the finished product tank is connected to the alkali hydrolysis rectification column through the second reflux pump, a lower part of the alkali hydrolysis rectification column is connected to the second reboiler through a circulation pipeline, and bottom of the alkali hydrolysis rectification column is connected to the second extraction pump.

For the sake of simplicity, device for distillation decolorization and purification of alcohol in maltol production described hereinafter is referred to as the present device.

The present device has a simple structure, is easy to use and has low cost of use.

For solving above-mentioned second technical problem, the invention provides a purification method using the above device for distillation decolorization and purification of alcohol in maltol production, comprising the following steps:
(1) pumping crude alcohol into the rectification column at a speed of 1.5 m³/h by the first feed pump, heating the crude alcohol in the rectification column by the first reboiler with temperature of 85-87° C. at the bottom of the rectification column and temperature of 76-78° C. at the top of the rectification column, condensing the material distilled from the rectification column by the first condenser and sending the material into the reflux tank, refluxing one part of the material in the reflux tank and extracting the other part of the material into the alkali hydrolysis kettle with a reflux ratio of 0.6, and extracting the waste at the bottom of the rectification column by the first extraction pump;
(2) adding caustic soda into the alkali hydrolysis kettle with amount of 25 kg caustic soda per 4 m³ material, increasing temperature up to 77-78° C., holding the temperature for 1 h to finish alkali hydrolysis, and sending the material into the alkali hydrolysis receiving tank;
(3) pumping the material in the alkali hydrolysis receiving tank into the alkali hydrolysis rectification column at a speed of 1 m³/h by the second feed pump, heating the material in the alkali hydrolysis rectification column by a second reboiler with temperature of 80-82° C. at the bottom of the alkali hydrolysis rectification column and temperature of 77-78° C. at the top of the alkali hydrolysis rectification column, condensing the material distilled from the rectification column by the second condenser and sending the material into the finished product tank, refluxing one part of the material in the finished product tank with a reflux ratio of 1.5, extracting qualified alcohol in the finished product tank after sampled and tested, and extracting the waste at the bottom of the alkali hydrolysis rectification column by the second extraction pump.

For the sake of simplicity, the purification method using the present device for distillation decolorization and purification of alcohol in maltol production described hereinafter is referred to as the present method.

The advantage of the present method: 1. the alcohol content after processing by the present method can reach more than 95%, and impurity content is low simultaneously; 2. the alcohol obtained by distilling has good transparency, has no peculiar smell, and is recovered to crystallization, which improves the odor quality of the finished maltol; 3. the alcohol after rectification can be sold out, which reduces the cost compared with the previous discharge into wastewater.

### BRIEF DESCRIPTION OF FIGURES

FIG. 1 is a schematic diagram of the process of the invention.

### DETAILED DESCRIPTION

### Embodiment One

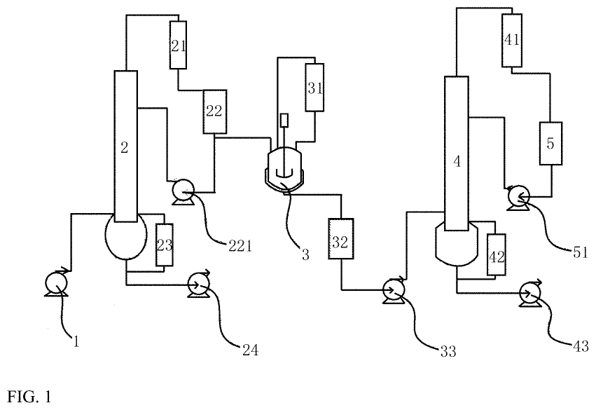

Referring to FIG. 1, a device for distillation decolorization and purification of alcohol in maltol production comprises a first feed pump 1, a rectification column 2, a first condenser 21, a reflux tank 22, a first reflux pump 221, a first reboiler 23, a first extraction pump 24, an alkali hydrolysis kettle 3, a second condenser 31, an alkali hydrolysis receiving tank 32, a second feed pump 33, an alkali hydrolysis rectification column 4, a third condenser 41, a finished product tank 5, a second reflux pump 51, a second reboiler 42, and a second extraction pump 43. The first feed pump 1 is connected to an inlet of the rectification column 2, the first condenser 21 is connected to an upper outlet of the rectification column 2, the other end of the first condenser 21 is connected to an inlet of the reflux tank 22, an outlet of the reflux tank 22 is connected to the rectification column 2 through the first reflux pump 221, a lower part of the rectification column 2 is connected to the first reboiler 23 through a circulation pipeline, bottom of the rectification column 2 is connected to the first production pump 24, an outlet of the reflux tank 22 is connected to an inlet of the alkali hydrolysis kettle 3 through the other pipeline, the alkali hydrolysis kettle 3 is connected to the second condenser 31 through a circulation pipeline, an outlet of the alkali hydrolysis kettle 3 is connected to an inlet of the alkali hydrolysis receiving tank 32, an outlet of the alkali hydrolysis receiving tank 32 is connected to an inlet of the alkali hydrolysis rectification column 4 through the second feed pump 33, an upper outlet of the alkali hydrolysis rectification column 4 is connected to the third condenser 41, the other end of the third condenser 41 is connected to an inlet of the finished product tank 5, an outlet of the finished product tank 5 is connected to the alkali hydrolysis rectification column 4 through the second reflux pump 51, a lower part of the alkali hydrolysis rectification column 4 is connected to the second reboiler 42 through a circulation pipeline, and bottom of the alkali hydrolysis rectification column 4 is connected to the second extraction pump 43.

### Embodiment Two

A purification method using the device for distillation decolorization and purification of alcohol in maltol production comprises the following steps:
(1) pumping crude alcohol into the rectification column at a speed of 1.5 m³/h by the first feed pump, heating the crude alcohol in the rectification column by the first reboiler with temperature of 85° C. at the bottom of the rectification column and temperature of 76° C. at the top of the rectification column, condensing the material distilled from the rectification column by the first condenser and sending the material into the reflux tank, refluxing one part of the material in the reflux tank and extracting the other part of the material into the alkali hydrolysis kettle with a reflux ratio of 0.6, and extracting the waste at the bottom of the rectification column by the first extraction pump;
(2) adding caustic soda into the alkali hydrolysis kettle with amount of 25 kg caustic soda per 4 m³ material, increasing temperature up to 77° C., holding the temperature for 1 h to finish alkali hydrolysis, and sending the material into the alkali hydrolysis receiving tank;
(3) pumping the material in the alkali hydrolysis receiving tank into the alkali hydrolysis rectification column at a speed of 1 m³/h by the second feed pump, heating the material in the alkali hydrolysis rectification column by a second reboiler with temperature of 80° C. at the bottom of the alkali hydrolysis rectification column and temperature of 77° C. at the top of the alkali hydrolysis rectification column, condensing the material distilled from the rectification column by the second condenser and sending the material into the finished product tank, refluxing one part of the material in the finished product tank with a reflux ratio of 1.5, extracting qualified alcohol in the finished product tank after sampled and tested, and extracting the waste at the bottom of the alkali hydrolysis rectification column by the second extraction pump.

After testing, the water content of the obtained ethanol is 0.6% and the impurity content is 3.2%.

### Embodiment Three

A purification method using the device for distillation decolorization and purification of alcohol in maltol production comprises the following steps:
(1) pumping crude alcohol into the rectification column at a speed of 1.5 m³/h by the first feed pump, heating the crude alcohol in the rectification column by the first reboiler with temperature of 86° C. at the bottom of the rectification column and temperature of 76° C. at the top of the rectification column, condensing the material distilled from the rectification column by the first condenser and sending the material into the reflux tank, refluxing one part of the material in the reflux tank and extracting the other part of the material into the alkali hydrolysis kettle with a reflux ratio of 0.6, and extracting the waste at the bottom of the rectification column by the first extraction pump;
(2) adding caustic soda into the alkali hydrolysis kettle with amount of 25 kg caustic soda per 4 m³ material, increasing temperature up to 77.5° C., holding the temperature for 1 h to finish alkali hydrolysis, and sending the material into the alkali hydrolysis receiving tank;
(3) pumping the material in the alkali hydrolysis receiving tank into the alkali hydrolysis rectification column at a speed of 1 m³/h by the second feed pump, heating the material in the alkali hydrolysis rectification column by a second reboiler with temperature of 81° C. at the bottom of the alkali hydrolysis rectification column and temperature of 77.5° C. at the top of the alkali hydrolysis rectification column, condensing the material distilled from the rectification column by the second condenser and sending the material into the finished product tank, refluxing one part of the material in the finished product tank with a reflux ratio of 1.5, extracting qualified alcohol in the finished product tank after sampled and tested, and extracting the waste at the bottom of the alkali hydrolysis rectification column by the second extraction pump.

After testing, the water content of the obtained ethanol is 0.8% and the impurity content is 3.1%.

### Embodiment Four

A purification method using the device for distillation decolorization and purification of alcohol in maltol production comprises the following steps:
(1) pumping crude alcohol into the rectification column at a speed of 1.5 m³/h by the first feed pump, heating the crude alcohol in the rectification column by the first reboiler with temperature of 87° C. at the bottom of the rectification column and temperature of 78° C. at the top of the rectification column, condensing the material distilled from the rectification column by the first condenser and sending the material into the reflux tank, refluxing one part of the material in the reflux tank and extracting the other part of the material into the alkali hydrolysis kettle with a reflux ratio of 0.6, and extracting the waste at the bottom of the rectification column by the first extraction pump;
(2) adding caustic soda into the alkali hydrolysis kettle with amount of 25 kg caustic soda per 4 m³ material, increasing temperature up to 78° C., holding the temperature for 1 h to finish alkali hydrolysis, and sending the material into the alkali hydrolysis receiving tank;
(3) pumping the material in the alkali hydrolysis receiving tank into the alkali hydrolysis rectification column at a speed of 1 m³/h by the second feed pump, heating the material in the alkali hydrolysis rectification column by a second reboiler with temperature of 82° C. at the bottom of the alkali hydrolysis rectification column and temperature of 78° C. at the top of the alkali hydrolysis rectification column, condensing the material distilled from the rectification column by the second condenser and sending the material into the finished product tank, refluxing one part of the material in the finished product tank with a reflux ratio of 1.5, extracting qualified alcohol in the finished product tank after sampled and tested, and extracting the waste at the bottom of the alkali hydrolysis rectification column by the second extraction pump.

After testing, the water content of the obtained ethanol is 1.0% and the impurity content is 3.0%.

The above described are only specific embodiments of the present application, under the above-mentioned teachings of the application, those skilled in the art can carry out other improvements or deformations on the basis of the above-mentioned embodiments. Those skilled in the art should understand that the above specific description is only to better explain the purpose of the present application, and the protection scope of the present application should be subject to the protection scope of the claims.

Furthermore, those skilled in the art can understand that although some embodiments described herein include some features included in other embodiments but not others, the combination of features of different embodiments means that they are within the scope of the present application and form different embodiments. For example, in the following claims, anyone of the claimed embodiments may be used in any combination.

What is claimed is:

1. A device for distillation decolorization and purification of alcohol in maltol production, is characterized in that, comprising a rectification column, an alkali hydrolysis kettle, and an alkali hydrolysis rectification column; an upper outlet of the rectification column is connected to an inlet of the alkali hydrolysis kettle, an outlet of the alkali hydrolysis kettle is connected to an inlet of the alkali hydrolysis rectification column, and an upper outlet of the alkali hydrolysis rectification column is connected to an inlet of a finished product tank.

2. The device for distillation decolorization and purification of alcohol in maltol production according to claim 1, is characterized in that, further comprising a first condenser, a second condenser, and a third condenser, the first condenser is connected to an upper outlet of the rectification column, the alkali hydrolysis tank is connected to the second condenser through a circulation pipeline, and an upper outlet of the alkali hydrolysis rectification column is connected to the third condenser.

3. The device for distillation decolorization and purification of alcohol in maltol production according to claim 2, is characterized in that, the other end of the first condenser other end is connected to an inlet of the reflux tank, an outlet of the reflux tank is connected to the rectification column through the first reflux pump, and a lower part of the rectification column is connected to the first reboiler through a circulation pipeline.

4. The device for distillation decolorization and purification of alcohol in maltol production according to claim 2, is characterized in that, the other end of the third condenser is connected an inlet of the finished product tank, an outlet of the finished product tank is connected to the alkali hydrolysis rectification column through the second reflux pump, a lower part of the alkali hydrolysis rectification column is connected to the second reboiler through a circulation pipeline, and bottom of the alkali hydrolysis rectification column is connected to the second extraction pump.

5. The device for distillation decolorization and purification of alcohol in maltol production according to claim 1, is characterized in that, further comprising a first feed pump, a first condenser, a reflux tank, a first reflux pump, a first reboiler, a first extraction pump, a second condenser, an alkali hydrolysis receiving tank, a second feed pump, a third condenser, a finished product tank, a second reflux pump, a second reboiler, and a second extraction pump, the first feed pump is connected to an inlet of the rectification column, the first condenser is connected to an upper outlet of the rectification column, the other end of the first condenser is connected to an inlet of the reflux tank, an outlet of the reflux tank is connected to the rectification column through the first reflux pump, a lower part of the rectification column is connected to the first reboiler through a circulation pipeline, bottom of the rectification column is connected to the first production pump, an outlet of the reflux tank is connected to an inlet of the alkali hydrolysis kettle through the other pipeline, the alkali hydrolysis kettle is connected to the second condenser through a circulation pipeline, an outlet of the alkali hydrolysis kettle is connected to an inlet of the alkali hydrolysis receiving tank, an outlet of the alkali hydrolysis receiving tank is connected to an inlet of the alkali hydrolysis rectification column through the second feed pump, an upper outlet of the alkali hydrolysis rectification column is connected to the third condenser, the other end of the third condenser is connected to an inlet of the finished product tank, an outlet of the finished product tank is connected to the alkali hydrolysis rectification column through the second reflux pump, a lower part of the alkali hydrolysis rectification column is connected to the second reboiler through a circulation pipeline, and bottom of the alkali hydrolysis rectification column is connected to the second extraction pump.

6. A purification method using the device for distillation decolorization and purification of alcohol in maltol production according to claim 1, is characterized in that, comprising the following steps:
(1) pumping crude alcohol into the rectification column at a speed of 1.5 m³/h by the first feed pump, heating the crude alcohol in the rectification column by the first reboiler with temperature of 85-87° C. at the bottom of the rectification column and temperature of 76-78° C. at the top of the rectification column, condensing the material distilled from the rectification column by the first condenser and sending the material into the reflux tank, refluxing one part of the material in the reflux tank and extracting the other part of the material into the alkali hydrolysis kettle with a reflux ratio of 0.6, and extracting the waste at the bottom of the rectification column by the first extraction pump;
(2) adding caustic soda into the alkali hydrolysis kettle with amount of 25 kg caustic soda per 4 m³ material, increasing temperature up to 77-78° C., holding the temperature for 1 h to finish alkali hydrolysis, and sending the material into the alkali hydrolysis receiving tank;
(3) pumping the material in the alkali hydrolysis receiving tank into the alkali hydrolysis rectification column at a speed of 1 m³/h by the second feed pump, heating the material in the alkali hydrolysis rectification column by a second reboiler with temperature of 80-82° C. at the bottom of the alkali hydrolysis rectification column and temperature of 77-78° C. at the top of the alkali hydrolysis rectification column, condensing the material distilled from the rectification column by the second condenser and sending the material into the finished product tank, refluxing one part of the material in the finished product tank with a reflux ratio of 1.5, extracting qualified alcohol in the finished product tank after sampled and tested, and extracting the waste at the bottom of the alkali hydrolysis rectification column by the second extraction pump.

7. A purification method using the device for distillation decolorization and purification of alcohol in maltol production is characterized in that, comprising the following steps:
Step 1: pumping crude alcohol into the rectification column with temperature of 85-87° C. at the bottom of the rectification column and temperature of 76-78° C. at the top of the rectification column, refluxing one part of the material distilled from the rectification column, extracting the other part of the material into the alkali hydrolysis kettle with a reflux ratio of 0.6;
Step 2: adding caustic soda into the alkali hydrolysis kettle, increasing temperature up to 77-78° C., and holding the temperature to finish alkali hydrolysis;
Step 3: sending the material processed by holding to finish alkali hydrolysis in step 2 to the alkali hydrolysis rectification column with temperature of 80-82° C. at the bottom of the alkali hydrolysis rectification column and temperature of 77-78° C. at the top of the alkali hydrolysis rectification column, refluxing one part of the material distilled from the alkali hydrolysis rectification column with a reflux ratio of 1.5.

8. A purification method using the device for distillation decolorization and purification of alcohol in maltol production according to claim 7, is characterized in that, comprising the following steps: the reflux ratio in step 1 is 0.6.

9. A purification method using the device for distillation decolorization and purification of alcohol in maltol production according to claim 7, is characterized in that, comprising the following steps: the reflux ratio in step 3 is 1.5.
